(19) United States
(12) Patent Application Publication
(10) Pub. No.: US 2016/0128982 A1
(43) Pub. Date: May 12, 2016

(54) ANTIMICROBIAL COMPOSITIONS AND METHODS OF USE

(71) Applicant: DERMCARE-VET PTY LTD, Slacks Creek, Queensland (AU)

(72) Inventors: Kenneth Vincent Mason, Mt Gravatt East (AU); Jacqueline Louise Wiley, Birkdale (AU); Sarika Namjoshi, Slack Creek (AU)

(21) Appl. No.: 14/901,703

(22) PCT Filed: Jun. 26, 2014
(86) PCT No.: PCT/AU2014/050095

§ 371 Date: Dec. 28, 2015

(30) Foreign Application Priority Data
Jun. 26, 2013 (AU) 2013902342

(51) Int. Cl. A61K 31/4174; A61K 31/155

(52) U.S. Cl. CPC A61K 31/4174; A61K 31/155

(57) ABSTRACT

Antimicrobial compositions include at least one antimicrobial azole compound, particularly imidazole, triazole or thiazole compounds, and at least one polymeric biguanide compound. The compositions are useful in treating or preventing microbial infections of the skin and epithelial lined body cavities, such as ears.

### FIELD OF THE INVENTION

The present invention relates to antimicrobial compositions comprising at least one antimicrobial azole compound, particularly imidazole, triazole or thiazole compounds, and at least one polymeric biguanide compound. Methods for their use in treating or preventing microbial infections of the skin and epithelial lined body cavities, such as ears, using the compositions of the invention are also described.

### BACKGROUND OF THE INVENTION

Treatment of infections of the skin and epithelial lined body cavities such as the external ear, in humans and other warm blooded animals, can be very difficult as these areas are exposed to the external environment and come into contact with many micro-organisms, such as bacteria and fungi, including antimicrobial resistant micro-organisms. Antimicrobial resistance may be developed as a consequence of repeated exposure to a suboptimal dose of an antimicrobial drug or because of repeated exposure during treatment of recurrent infections and subsequent selection of resistant strains, or may be a result of invasion by a micro-organism which has inherent antimicrobial resistance.

The commonly encountered micro-organisms that infect the skin and cavities such as external ears in mammals include the bacteria Staphylococcus spp., Enterobacteriaceae spp. such as Escherichia coli, Klebsiella spp., and Proteus spp. such as Proteus mirabilis, Proteus vulgaris and Pseudomonads such as Pseudomonas aeruginosa. In some cases, the bacteria may be present together with fungi such as Malassezia pachydermatis or Candida albicans. These micro-organisms may thrive in cavities such as ear canals and skin intertriginous zones such as the auxiliary fossa (armpit), and sometimes even benefit from antimicrobial treatment, possibly by removal of other susceptible micro-organisms competing for the same environment. An example of such a phenomenon is the dramatic overgrowth of Malassezia spp. such as Malassezia pachydermatis after reduction in Pseudomonas numbers (Foster, DeBoer, 1998, The role of Pseudomonas in canine ear disease, Compendium on Continuing Education, 20 (8), 909-918).

Purulent exudates found in skin infections and particularly ear infections often contain inflammatory cells, biological proteins, enzymes, DNA and other biological compounds which inactivate the antimicrobial and biological action of drugs prescribed for treatment of the infections. In the case of ear infections, many drugs that are prescribed are neuro-toxic and are thus ototoxic (Rohn et al. 1993, Ototoxicity of Topical Agents, Otolaryngology Clinics of North America, 26(5), 2167-2169).

There is a need for simple and effective treatment for skin infections that is rapid and active at low levels, below toxic levels. There is also a need for treatments that have a broad action in reversing the resistance spectrum of an infection to antimicrobial drugs.

Azole antifungal agents, such as miconazole, are known for topical use on skin and mucus membranes to control fungal infections such as thrush, athlete's foot and ringworm. Although azole compounds may have good antifungal properties, they are known to have limited antibacterial properties.

Biguanide compounds are known antiseptics and have been used as topical antiseptics, in contact lens solutions and as disinfectants. Polymeric biguanides such as Polyhexamethylene biguanide (PHMB) and others are described by East et al. 1997 (Polymer, 38 (15), 3973-3984) and Ikeda et al., 1984 (Antimicrobial Agents and Chemotherapy, 26(2), 139-144).

### SUMMARY OF THE INVENTION

Advantageously, the present inventor has found that a combination of at least one azole compound and at least one polymeric biguanide demonstrate a synergistic effect in treating microbial infections including bacterial and fungal infections and mixtures of bacterial and fungal infections.

In a first aspect of the invention, there is provided a pharmaceutical or veterinary composition comprising at least one azole compound or a pharmaceutically acceptable salt thereof and at least one polymeric biguanide compound or a pharmaceutically acceptable salt thereof.

In another aspect of the invention, there is provided a method of treating or preventing infections of the skin or an epithelial lined cavity in a mammal, comprising topical administration of an effective amount of a composition comprising at least one azole compound or a pharmaceutically acceptable salt thereof and at least one polymeric biguanide compound or a pharmaceutically acceptable salt thereof.

In yet another aspect of the invention, there is a use of a composition comprising at least one azole compound or a pharmaceutically acceptable salt thereof and at least one polymeric biguanide compound or a pharmaceutically acceptable salt thereof; in the manufacture of a medicament for treating or preventing infections of the skin or an epithelial lined cavity in a mammal.

### BRIEF DESCRIPTION OF THE FIGURES

FIG. 1A shows an agar plate inoculated with S. pseudintermedius and the result of zone inhibition according to the invention.

FIG. 1B shows an agar plate inoculated with M. pachydermatis and the result of zone inhibition according to the invention.

### DESCRIPTION OF THE INVENTION

The compositions and methods of the present invention are useful for inhibiting or treating infections of the skin or other epithelial lined body cavities, such as external ears, in mammals.

In some embodiments, the at least one azole compound is an imidazole, triazole or thiazole compound or a mixture thereof. Suitable imidazole compounds include but are not limited to, bifonazole, butoconazole, clotrimazole, enilconazole, econazole, fenticonazole, isoconazole, ketoconazole, miconazole, omoconazole, oxiconazole, sertaconazole, sulconazole, thiabendazole, tioconazole, Elubiol (dichlorophenyl imidazoldioxolan) and mixtures thereof. Suitable triazole compounds include but are not limited to, albaconazole, fluconazole, isavuconazole, itraconazole, posaconazole, ruvaconazole, terconazole and voriconazole. Suitable thiazole compounds include but are not limited to abafungin. In particular embodiments, the azole compound is selected from miconazole and ketaconazole.

In some embodiments, the polymeric biguanide is one in which the biguanide moiety appears in the polymer backbone, such as those described by East et al. 1997. In some embodiments, the polymeric biguanide has the formula:

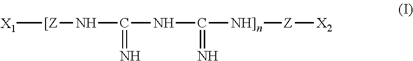

or a tautomer thereof, wherein Z is absent or an organic divalent bridging group and each Z may be the same or different throughout the polymer; n is at least 3, preferably 5 to 20 and X₁ and X₂ are independently selected from —NH₂, —NH—C(═NH)—NH—CN, optionally substituted alkyl, optionally substituted cycloalkyl, optionally substituted aryl, optionally substituted heterocyclyl and optionally substituted heteroaryl; or a pharmaceutically or veterinary acceptable salt thereof. In particular embodiments, the molecular weight of the polymeric biguanide compound is at least 1,000 amu, especially between 1,000 amu and 50,000 amu. In a single composition, n may vary providing a mixture of polymeric biguanides. In some embodiments, the polymeric biguanides have a mean molecular weight in the region of 2,900 to 15,000, especially 3,000 to 8,000, a particularly 3,200 to 5,000, especially 3,500 to 4,500.

The above polymeric biguanide compounds and methods for their preparation are described in, for example, U.S. Pat. No. 3,428,576 and East et. al., 1997 (Polymer, 38(15), 3973-3984).

In particular embodiments, the polymeric biguanides of formula (I) are polymeric hexamethylene biguanides, such as polyhexanide or PHMB (commercially available as Vantocil®, Baquacil®, Arlagard®, Lonzabac BG® and Cosmocil®) of the following formula:

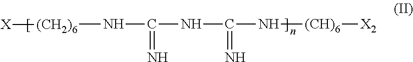

or a tautomer thereof, wherein n is an integer from 3 to 500 and X₁ and X₂ are independently selected from —NH₂, —NH—C(═NH)—NH—CN, optionally substituted alkyl, optionally substituted cycloalkyl, optionally substituted aryl, optionally substituted heterocyclyl and optionally substituted aryl or a pharmaceutically or veterinary acceptable salt thereof. In particular embodiments, n has an average value of 3 to 15, especially 3 to 12, more especially the polymeric hexamethylene biguanides, commercially available, for example, as the hydrochloride salt, from Avecia (Wilmington, Del., USA) under the trademark Cosmocil CQ®.

In another embodiment of the invention, the polymeric biguanides are fractionated polymeric biguanides where the lower molecular weight proportion of the polymer is removed. In particular embodiments, the polymeric biguanide compositions used in the compositions of the invention have a fraction of polymers having a value of n≦5at less than 2 wt %, especially less than 0.5 wt % and most especially less than 0.1 wt %.

In other embodiments, the polymeric biguanides are those with pendant biguanide groups having the formula:

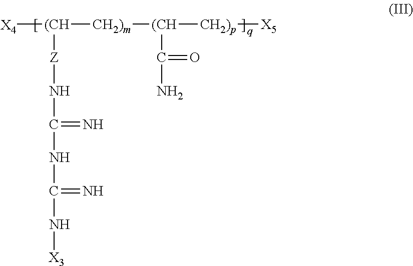

or a tautomer thereof, wherein X₃ is selected from —NH₂, —NH—C(═NH)—NH—CN, optionally substituted alkyl, optionally substituted cycloalkyl, optionally substituted aryl, optionally substituted heterocyclyl and optionally substituted aryl or a pharmaceutically or veterinary acceptable salt thereof, X₄ and X₅ are independently selected from H or X₃, Z is absent or is a divalent bridging group, m is an integer from 1 to 10, p is 0 or an integer from 1 to 10 and q is an integer from 1 to 1000.

Exemplary polymeric biguanides having pendant biguanide groups are those described by Ikeda et. al., 1984 (Antimicrobial Agents and Chemotherapy, 26(2), 139-144) in which X₄ and X₅ are hydrogen, Z is —C(O)—O—CH₂CH₂—C₆H₄—, X₃ is phenyl or optionally substituted phenyl, especially 4-chlorophenyl or 3,4-dichlorophenyl, m is an integer from 1 to 10, p is 0, q is an integer from 1 to 500 (homopolymer) or where m is 1 to 10, p is 1 to 10 and q is 1 to 500 (co-polymer with acrylamide). The polymeric biguanides having pendant biguanide groups and methods for their preparation are described in Ikeda et. al., 1984 (ibid).

As used herein, the term “tautomer” refers to isomeric forms of a compound which have migration of a hydrogen atom accompanied by movement of adjacent double bonds. For example, in the formulae set out above, the biguanide moiety may tautomerise to provide different isomers according to the following:

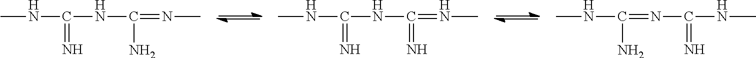

As used herein, the term “alkyl” refers to monovalent, straight chain or branched hydrocarbon groups, having 1 to 10 carbon atoms as appropriate. For example, suitable alkyl groups include, but are not limited to, methyl, ethyl, propyl, isopropyl, n-butyl, sec-butyl, tert-butyl, pentyl, 2-methylpentyl, 3-methylpentyl, n-hexyl, 2-, 3- or 4-methylhexyl, 2-, 3- or 4-ethylhexyl, heptyl, octyl, nonyl and decyl.

As used herein, the term “cycloalkyl”, refers to saturated and unsaturated cyclic hydrocarbon groups. Suitable cycloalkyl groups include, but are not limited to, cyclopropyl, cyclobutyl, cyclopentyl, cyclohexyl and cyclohexadienyl

The term “aryl” as used herein, refers to C₆-C₁₀ aromatic hydrocarbon groups such as phenyl and naphthyl.

The term “heterocyclyl” or “heterocyclic”, as used herein, refers to saturated or unsaturated monocyclic, polycyclic, fused or conjugated cyclic hydrocarbon residues, preferably C₃₋₆, wherein one or more carbon atoms (and where appropriate, hydrogen atoms attached thereto) are replaced by a heteroatom so as to provide a non-aromatic residue. Suitable heteroatoms include O, N and S. Where two or more carbon atoms are replaced, this may be by two or more of the same heteroatom or by different heteroatoms. Suitable examples of heterocyclic groups may include pyrrolidinyl, pyrrolinyl, piperidyl, piperazinyl, morpholino, indolinyl, imidazolidinyl, pyrazolidinyl, thiomorpholino, dioxanyl, tetrahydrofuranyl, tetrahydropyranyl, tetrahydropyrrolyl and the like.

The term “heteroaryl” or “heteroaromatic”, used herein, represents a stable monocyclic or bicyclic ring of up to 6 atoms in each ring, wherein at least one ring is aromatic and contains from 1 to 4 heteroatoms selected from the group consisting of O, N and S. Heteroaryl groups within the scope of this definition include, but are not limited to, acridinyl, carbazolyl, cinnolinyl, quinoxalinyl, pyrazolyl, indolyl, benzotriazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl and tetrahydroquinoline.

Alkyl, cycloalkyl, heterocyclyl, heteroaryl and aryl groups of the invention may be optionally substituted with 1 to 5 groups selected from OH, OC₁₋₆alkyl, Cl, Br, F, I, NH₂, NH(C₁₋₆alkyl), N(C₁₋₆alkyl)₂, SH, SC₁₋₆alkyl, CO₂H, CO₂C₁₋₆alkyl, CONH₂, CONH(C₁₋₆alkyl) and CON(C₁₋₆alkyl)₂.

As used herein, the term “divalent bridging group” refers to a radical that has a valence of two and is able to bind with two other groups. Examples of suitable divalent bridging groups include but are not limited to —(CH₂)ₜ— where t is an integer from 1 to 10, —O—, —S—, a divalent saturated or aromatic carbocyclic ring or a heterocyclic or heteroaromatic ring or a combination of such divalent and/or cyclic moieties. For example a saturated C₆ cyclic group would include —C₆H₁₀—, a C₆ aromatic group would include —C₆H₄—, a C₆ heterocyclic group would include

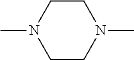

and a C₆ heteroaromatic would include

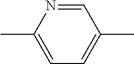

Other divalent bridging groups include alkylene groups (—CH₂—)ₜ in which one or more carbon atoms have been replaced by NH, S, O,

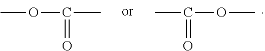

In a preferred embodiment the divalent bridging group is —(CH₂)ₜ— where t is an integer from 1 to 10, especially 1 to 6, more especially 6.

Suitable pharmaceutically or veterinary acceptable salts include, but are not limited to, salts of pharmaceutically or veterinary acceptable inorganic acids such as hydrochloric, sulphuric, phosphoric, nitric, carbonic, boric, sulfamic, sulfonic and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methane sulphonic, toluene sulphonic, benzene sulphonic, salicylic, sulphanilic, aspartic, glutamic, edetic, stearic, palmitic, oleic, lauric, pantothenic, tannic, ascorbic and valeric acids. Preferred salts include salts of hydrochloric, boric, sulfonic, acetic, gluconic, citric and tartaric acids.

The amount of azole compound present in the composition may depend on the micro-organism or combination or micro-organisms being treated. In some embodiments, the azole compound is present in the pharmaceutical or veterinary composition in an amount of between 0.00001 wt % and 2.0 wt %, especially 0.00001 wt % to 0.1 wt %, more especially 0.00001 to 0.01 wt % or 0.00001 wt % to 0.001 wt %. The azole compound may be present in the composition in an amount which has little or no antimicrobial effect when used alone, but when used in the combination of the invention, exerts an antimicrobial effect. That is, the effective amount may act synergistically with the polymeric biguanide. For example, an azole such as miconazole begins to have an antimicrobial effect on bacteria such as Staphylococcus pseudintermedius at about 0.00012 wt % of the composition. However, when combined with a polymeric biguanide, the azole demonstrates an antimicrobial effect below this amount, for example, in the range of 0.00003 wt % to 0.00012 wt %. In the case of a fungal infection, such as an infection caused by C. albicans, an azole may begin to have an antimicrobial effect at about 0.0009 wt % of the composition when used alone. However, when combined with a polymeric biguanide, the azole demonstrates an antimicrobial effect below this amount, for example, in the range of 0.00012 wt % to 0.0009 wt % of the composition.

The amount of azole compound present may also be determined by the type of formulation being used. For example, an ointment, lotion or wash (lavage) formulation may contain the amounts set out above. However, the formulation of a shampoo composition may require greater amounts, for example, 0.1 to 4.0 wt %, especially about 1 to 2 wt % of the composition.

The amount of polymeric biguanide present in the composition may also depend on the micro-organism or combination or micro-organisms being treated. In some embodiments, the polymeric biguanide is present at a concentration in the range of 0.0001 wt % to 5 wt %, especially 0.0001 wt % to 3.0 wt %, more especially 0.0001 to 0.5 wt %, for example 0.0001 to 0.1 wt %. In some embodiments, the amount of polymeric biguanide used in the composition is an amount which has little or no antimicrobial activity when used alone, but when used in the combination of the invention, exerts an antimicrobial effect. That is, the effective amount may act synergistfcally with the azole compound. For example, a polymeric biguanide such as PHMB begins to exert an antimicrobial effect on bacteria such as Staphylococcus pseudintermedius at a concentration of about 0.00024 wt % of the composition. However, when combined with an azole compound, the polymeric biguanide demonstrates an antimicrobial effect below this amount, for example, in the range of 0.00003 wt % to 0.00024 wt %. In the case of a fungal infection, such as an infection caused by C. albicans, a polymeric biguanide may begin to have an antimicrobial effect at about 0.0156 wt % of the composition when used alone. However, when combined with an azole compound, the polymeric biguanide demonstrates an antimicrobial effect below this amount, for example, in the range of 0.003 wt % to 0.0156 wt % of the composition.

The amount of polymeric biguanide present may also be determined by the type of formulation being used. For example, an ointment, lotion or wash (lavage) formulation may contain the amounts set out above. However, the formulation of a shampoo composition may require greater amounts, for example, 1.0 to 3.0 wt %, especially about 2 wt % of the composition.

In some embodiments, the ratio of azole compound to polymeric biguanide compound in the composition is in the range of 1:1 to 1:300, for example, 1:1 to 1:150, 1:1 to 1:100, 1:1 to 1:80 or 1:1 to 1:70. The ratio of azole compound to polymeric biguanide in the composition may vary depending on the micro-organism being treated. For example, the ratio of azole compound to polymeric biguanide that may be used for treating a bacterial infection such as one caused by Staphylococcus pseudintermedius may be in the range of 1:1 to 1:10, for example, about 1:4. The ratio of azole compound to polymeric biguanide that may be used in treating a fungal infection, such as one caused by C. albicans, may be in the range of 1:120 to 1:175, for example, 1 to 145 to 1:150, especially about 1:148.

In some embodiments, the amounts of azole compound and polymeric biguanide are synergistic amounts, that is, are amounts that provide a synergistic effect. This allows the azole compound to be used in low amounts, thereby reducing toxicity in the mammal and reducing the likelihood of development of resistance in the micro-organism.

In some embodiments, the pharmaceutical or veterinary composition may further include an additive which enhances the antimicrobial activity of the composition. Such additives include propylene glycol, glycerin, polypropylene glycol(s), polyethylene glycol(s), an antibiotic or a mixture of propylene glycol and/or polypropylene glycol(s) and/or polyethylene glycol(s) and/or glycerin and/or an antibiotic.

Suitable antibiotics include, but are not limited to, fluoroquinolones such as ciprofloxacin, norfloxacin, ofloxacin, enoxacin, perfloxacin, fleroxacin, enrofloxacin, marbofloxacin, sarafloxacin, orbifloxacin, danofloxacin; aminoglycosides such as streptomycin, netilmicin, kanamycin, neomycin, tobramycin, amikacin, sisomicin, ribostamycin, dibekacin, framycetin and gentamycin, penicillins and amino penicillins such as penicillin, ampicillin, amoxacillin, nafcillin, oxacillin and ticarcillin, cephalosporins such as ceftriaxone, cephalexin, cefadroxil and ceftiofur, B-lactams such as clavulanic acid, macrolides such as clarithromycin and erythromycin and other antibiotics such as dactinomycin, clindamycin, nalidixic acid, chloramphenicol, rifampicin, clofazimine, spectinomycin, polymyxin B, colistin, minocycline, vancomycin, hygromycin B or C, fusidic acid, trimethoprim and cefotaxime.

While in its simplest form the composition of the invention may be used neat as a combination of azole compound and polymeric biguanide or as an aqueous composition consisting of a polymeric biguanide and azole compound in water, the composition may also include other pharmaceutically acceptable or veterinary acceptable additives, such as surfactants, carriers, diluents and excipients.

Topical administration according to the invention may be by means of a liquid or vaporised composition. Suitable liquid compositions include lotions, ointments and gels and include aqueous solutions. Suitable vaporised compositions include sprays and aerosols. In some embodiments, the topical administration is administration of a liquid composition by lavage or by spray, such as that delivered by a trigger spray bottle. Other suitable means of application are known in the art, for example, a moistened gauze, swab, cotton, foam, sponge or cloth. In particular embodiments where the composition is to be topically applied to the skin, the composition may be in the form of a lotion, ointment, mousse or gel. In other embodiments, where the composition is to be topically applied to skin bearing hair, the composition may be in the form of a shampoo. Shampoos are particularly useful for application to the scalp of a human or to any body part or all body parts bearing hair, of an animal.

When the skin to be treated is in an epithelial lined body cavity, such as an ear, the composition may be a liquid or aqueous composition that is applied by lavage or by spray, such as that delivered by a trigger spray bottle. Of particular benefit is the physical flush effect that disrupts encrusted purulent and waxy accumulated material away from the ear lining, breaking it up and allowing penetration of other medications and flushing it from the ear canal.

Such a liquid flushing composition may be applied using a flush applicator. Suitable carriers for use in topical compositions include, but are not limited to, mineral oil, propylene glycol, polyethylene glycols, polyoxyethylene, polyoxypropylene, emulsifying wax, sorbitan monostearate, polysorbate 20, polysorbate 60, cetyl esters, wax, cetearyl alcohol, 2-octyldodecanol, benzyl alcohol, surfactants and water. In particular embodiments, the carrier is water.

Suitable carriers, excipients and diluents, where appropriate, include solvents, for example to solubilize the azole compound, dispersion agents, preservatives, penetration agents, surfactants, viscosity adjusters, anti-inflammatory agents, isotonic and absorption agents and the like. The choice of solvent used may be dictated by the site of application, for example, some solvents, such as various alcohols (eg. benzyl alcohol or benzaldehyde), should not be topically administered to the ear canal due to the irritant effect on a sensitive tympanum and/or canal denuded of its protective lining.

In some embodiments, the composition comprises a non-ionic, cationic, anionic or amphoteric surfactant or a combination thereof. Suitable surfactants include, but are not limited to, polysorbates, alkoxyphenol ethoxylates, glycosides, glycoside alkyl ethers, quaternary ammonium compounds, fatty acid sulfates, fatty ether sulfates, polyethoxylated glycolipids, polyethoxylated monoglycerides, poloxamines and betaines. In particular embodiments, the surfactants are selected from the group consisting of nonoxynol, octoxynol, phospholipids, polysorbate 20, polysorbate 80 and cocamidopropyl betaine. The surfactant will generally be present in a topical composition such as an ointment, lotion, gel, mousse or aqueous composition at a concentration of 0.001% w/w (10 ppm) to 5% w/w of the composition, preferably 0.01 to 2% w/w, most preferably 0.1 to 1% w/w, especially about 0.2% w/w. In some embodiments, the surfactant is in the range of about 0.01 to 0.02% w/w of the composition, especially about 0.015% w/w of the composition. In compositions such as shampoos, high proportions of surfactant or surfactant mixtures are included to achieve cleaning, foaming, viscosity and conditioning. The surfactants used may include fatty acid sulfates such as ammonium lauryl sulfate and sodium lauryl sulfate, fatty ether sulfates such as sodium laureth sulfate, polysorbates such as polysorbate 20 and polysorbate 80, glycosides such as deacylglycosides and aryl glycosides and betaines such as cocoamido propyl betaine. the surfactant will generally be present in the shampoo or other cleansing formulation in amounts that vary from <5% w/w of the composition up to 70% w/w of the composition. The amount of surfactants present will be determined by the cleansing required, for example, amounts of skin sebum and waxy oils present on skin of the subject and the other components in the composition such as bacterial or fungal products.

Suitable agents that enhance penetration into exudates and waxes or through the epithelial lining (epidermis and dermis) include, but are not limited to, dimethyl sulfoxide, polyvinyl pyrrolidone and light oils such as isopropyl myristate. Light oils also dissolve the oily waxes produced by ears and this is a valuable effect.

Suitable anti-inflammatory agents include, but are not limited to, corticosteroids such as prednisolone, triamcinolone, dexamethasone, betamethasone and momethaxone and suitable non-steroidal anti-inflammatory drugs include, but not limited to, ibuprofen, ketoprofen, suprofen, caprofen, meloxicam, tolfenamic acid, piroxicam, firocoxib and ketorolac. Suitable anti-allergic drugs include, but not limited to, cromolyn, emedastine, olopatadine and cyclosporine.

Suitable viscosity enhancers include, but are not limited to, propylene glycol, polyethylene glycol, polypropylene glycol(s), bentonite, celluloses such as methylcellulose, ethylcellulose and carboxymethylcellulose, and tragacanth.

The composition may also include a preservative. Many preservatives and mixtures thereof are known to those skilled in the art. Suitable preservatives include sodium benzoate, alpha-tocopherol, ascorbic acid, carotinoids, sorbic acid, benzoic acid, methyl paraben, ethyl paraben, propyl paraben, butyl paraben, isothiazolinones, propyl gallate, tertiary butylhydroquinone, butylated hydroxyanisole, butylated hydroxytoluene, sodium metabisulfite and sodium bisulfite. In some embodiments, the preservative is methyl paraben, propyl paraben or mixtures thereof.

The compositions described above are used in methods of treating infections of the skin or an epithelial lined body cavity in a mammal.

Suitable mammals include any mammal prone to skin infections, including humans, domestic animals such as pets, agriculturally useful animals, such as sheep, cattle, pigs and horses and captive wild animals, such as those kept in zoos. Particularly preferred mammals are humans and pets, such as horses, cats and dogs.

In particular embodiments, the compositions are used to treat skin infections, especially skin infections in humans or animals such as dogs. In other embodiments where the infection is an external ear infection, especially preferred mammals are humans and dogs, especially dogs such as long-haired, pendulous-eared breeds of dog. As used herein, the term “external ear” refers to the pinna or auricle and the auditory canal or meatus of the ear. In yet other embodiments, the epithelial lined body cavity is a respiratory tract such as a nasal cavity or nostril or a urogenital tract such as vaginal, uterine or urine associated cavities.

In some embodiments, the infection is a bacterial infection. In other embodiments, the infection is a fungal Infection. In yet other embodiments, the infection is caused by a mixture of fungi and bacteria that co-exist on the skin. The infection may be caused by Gram positive bacteria, Gram negative bacteria or fungi, such as yeast for example malassezia sp. or a mycelial type of fungi such as a dermatophyte.

In some embodiments, the infection is caused by bacteria selected from one or more of a Staphylococcus spp., a Streptococcus spp. a Enterobacteriaceae spp., a Klebsiella spp., a Proteus spp., and Pseudomonads. In particular embodiments, the bacteria may be selected from one or more of Staphylococcus intermedius, Staphylococcus pseudintermedius, Staphylococcus aureus, Escherichia coli, Proteus mirabilis, Proteus vulgaris and Pseudomonas aeruginosa. In some embodiments, the fungal infection is caused by a fungus such as yeast, an opportunistic environmental fungus such as Aspergillus spp. or a dermatophyte. In particular embodiments, the yeast may be a Candida spp. such as Candida albicans or a Malassezia spp. such as Malassezia pachydermatis and the Aspergillus spp. may be selected from Aspergillus clavatus, Aspergillus fischerianus, Aspergillus flavus and Aspergillus fumigatus. The dermatophyte may be selected from a Microsporum spp., an Epidermophyton spp. or a Trichophyton spp. including, but not limited to, Tinea pedis, Tinea cruris, Tinea corpora, Tinea faciei, Tinea capitis, Tinea manuum, Trichophyton rubrum, Trichophyton mentagrophytes, Trichophyton verrucosum, Trichophyton tonsurans, Trichophyton equinum, Trichophyton kanei, Trichophyton raubitschekii, Trichophyton violaceum, Epidermophyton floccosum, Microsporum audouinii, Microsporum canis, Microsporum equinum, Microsporum nanum and Microsporum versicolor.

The composition of the invention may be used in any amount that is effective to inhibit or treat or prevent the infection. As used herein, the term “effective amount” relates to an amount of the composition which, when administered according to a desired dosing regimen, provides the desired therapeutic activity or infection prevention. Dosing may occur at intervals of minutes, hours, days, weeks, months or years. An inhibiting effective amount is an amount of the composition, which when administered according to the desired dosage regimen, is sufficient to prevent the multiplication of microbes responsible for infection. A therapeutic effective amount or treatment effective amount is an amount of the composition, which when administered according to a desired dosage regimen, is sufficient to at least partially attain the desired therapeutic effect, or delay the onset of, or inhibit the progression of, halt, partially or fully the onset or progression of the infection or is able to reverse or partially reverse the antibiotic sensitivity of the organisms including lowering the minimum inhibitory concentration (MIC) or inducing a synergistic interaction. A preventative, effective amount of the composition, which when administered according to a desired dosage regimen, is sufficient to at least partially prevent or delay the onset of the infection.

Suitable dosage amounts and dosing regimens may be determined by the attending physician or veterinarian and may depend on the severity of the infection as well as the general age, health and weight of the subject being treated.

In some embodiments, the method may comprise the further step of sequentially or simultaneously administering an antibiotic or an anti-inflammatory agent. The simultaneous administration may be in the one composition or in separate compositions. Suitable antibiotics include, but are not limited to, fluoroquinolones such as ciprofloxacin, norfloxacin, ofloxacin, enoxacin, perfloxacin, fleroxacin, enrofloxacin, marbofloxacin, sarafloxacin, orbifloxacin, danofloxacin; aminoglycosides such as streptomycin, netilmicin, kanamycin, neomycin, tobramycin, amikacin, sisomicin, ribostamycin, dibekacin, framycetin and gentamycin, penicillins and amino penicillins such as penicillin, ampicillin, amoxacillin, nafcillin, oxacillin and ticarcillin, cephalosporins such as ceftriaxone, cephalexin, cefadroxil and ceftiofur, B-lactams such as clavulanic acid, macrolides such as clarithromycin and erythromycin and other antibiotics such as dactinomycin, clindamycin, nalidixic acid, chloramphenicol, rifampicin, clofazimine, spectinomycin, polymyxin B, colistin, minocycline, vancomycin, hygromycin B or C, fusidic acid, trimethoprim and cefotaxime. Suitable anti-inflammatory agents include, but are not limited to, corticosteroids such as prednisolone, triamcinolone, dexamethasone, betamethasone and mometasone and suitable non-steroidal anti-inflammatory drugs include, but not limited to, ibuprofen, ketoprofen, suprofen, caprofen, meloxicam, tolfenamic acid, piroxicam, firocoxib and ketorolac. Suitable anti-allergic drugs include, but not limited to, antihistamines, cromolyn, emedastine, olopatadine and cyclosporine.

In some embodiments, the method is used to inhibit or treat an infection of the external ear, especially the external ear of a dog. The ear canal of the modern dog that is long and convoluted and is therefore not self-cleaning. Dogs are unable to keep their ears sufficiently clean to prevent infection. Dogs that are particularly susceptible to ear infection tend to have long hair and pendulous ears.

A further difficulty with treating ear infections in dogs is that once drops or liquid has been placed in the ear canals, it is a reflex to shake their head to remove the liquid. It is important to have an ear wash or composition that will act rapidly upon administration to the ear canal.

In other embodiments, the method is used to treat a skin infection, especially a skin infection in a dog. When treating skin infections, rapid action may also be required as animals may shake off or lick off the composition shortly after application or it may be difficult for the composition to penetrate into some animal coat types, for example dense coat types.

In some embodiments, the composition is in the form of a shampoo, ear drops or an ear wash composition.

Those skilled in the art will appreciate that the invention described herein is susceptible to variations and modifications other than those specifically described. It is to be understood that the invention includes all such variations and modifications which fall within the spirit and scope.

The invention will now be described with reference to the following examples which are included for the purpose of illustration only and are not intended to limit the generality of the invention hereinbefore described.

### EXAMPLES

### Example 1

### Determination of Synergism Using Well Dilution Method

1. To determine whether propylene glycol was a suitable solvent to solubilize miconazole. A Mueller-Hinton agar (MH) plate containing a well was inoculated with Staphylococcus pseudintermedius and a Sabouraud dextrose agar (SAB) plate containing a 6 mm well was inoculated with Malassezia pachydermatis. The wells were filled with 50 μL propylene glycol and the MH plate were incubated at 37° C. for 18 to 24 hours and the SAB plate was incubated at 30 to 35° C. for 72 hours. The plates were then observed for any inhibition of microbial growth around the well.

No zone of inhibition was observed therefore propylene glycol did not have an antimicrobial effect on S. pseudintermedius or M. pachydermatis. Propylene glycol was a suitable solvent to solubilize miconazole.

2. To determine suitable concentrations of miconazole and PHMB, MH agar plates containing wells were inoculated with S. pseudintermedius and SAB plates containing wells were inoculated with M. pachydermatis. The wells were filled with 50 μL of varying concentrations of miconazole or PHMB. The MH plates were incubated at 37° C. for 18 to 24 hours and the SAB plates were incubated at 30 to 35° C. for 48 hours.

After incubation, the diameter of the zone of inhibition around the well was measured. The inhibition zones are shown in Table 1.

TABLE 1

| | PHMB | Miconazole | Zone of Inhibition |
|---|---|---|---|
| Microbe | concentration | concentration | (mm) |
| S. pseudintermedius | 0.125% | — | 17-18 |
| S. pseudintermedius | 0.0625% | — | 14 |
| S. pseudintermedius | — | 0.0009% | 14-17 |
| S. pseudintermedius | — | 0.0019% | 14-16 |
| S. pseudintermedius | — | 0.0039% | 17-18 |
| M. pachydermatis | 0.125% | — | 24-27 |
| M. pachydermatis | 0.25% | — | 27-28 |
| M. pachydermatis | — | 0.0009% | 25 |

Concentrations of PHMB and miconazole that had similar sizes of zones of inhibition were selected for determination of synergy.

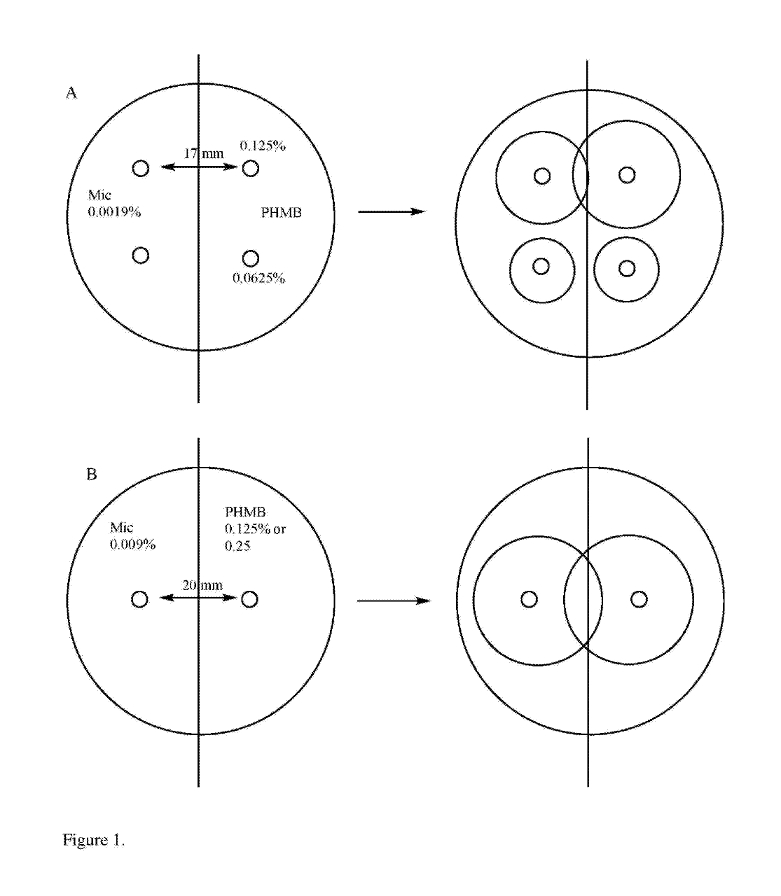

3. To determine whether the combinations of miconazole and PHMB had a synergistic effect, MH and SAB agar plates with four or two wells at varying distances apart were inoculated with S. pseudintermedius and M. pachydermatis respectively. The wells on one side of the plate were dosed with 50 μL miconazole and the other side of the plate with 50 μL PHMB at varying concentrations as shown in FIG. 1.

FIG. 1A shows an agar plate inoculated with S. pseudintermedius. The plate contains 4 wells. The wells on the left hand side of the plate were 17 mm apart from the wells on the right hand side of the plate. The wells on the left hand side of the plate were dosed with 50 μL 0.0039% miconazole. The top well on the right hand side of the plate was dosed with 50 μL 0.125% PHMB and the bottom well was dosed with 50 μL 0.0625% PHMB. The plates were incubated for 18 to 24 hours at 37° C. After incubation the plates were examined for a zone of inhibition between two upper or two lower wells. As shown in FIG. 1A, a zone of inhibition formed between the wells having 0.0039% miconazole and 0.125% PHMB demonstrating synergy between the miconazole and PHMB. No zone of inhibition was observed between the wells having 0.0039% miconazole and 0.625% PHMB.

FIG. 1B shows an agar plate inoculated with M. pachydermatis. Two plates were used and the wells were placed centrally on the left and right sides of the plates 20mm apart. The well on the left hand side of both plates was dosed with 50 μL 0.0009% miconazole. On one plate, the right hand well was dosed with 50 μL 0.125% PHMB and on the other plate the right hand well was dosed with 50 ∞L 0.25% PHMB. The plates were incubated for 48 hours at 30 to 35° C. After incubation the plates were examined for a zone of inhibition between the left and right hand wells. As shown in FIG. 1B, a zone of inhibition formed between the wells on both plates demonstrated synergy between the miconazole and both concentrations of PHMB.

### Example 2

### Determination of Synergism Using Minimum Inhibitory Concentration (MIC)

The MIC of PHMB, miconazole and mixtures of PHMB and miconazole were determined by adding varying concentrations of each component or their combination to molten Mueller-Hinton agar and SAB agar. The miconazole was initially dissolved in propylene glycol but dilutions were prepared by diluting this solution with water.

The concentrations of miconazole, PHMB or combination of PHMB and miconazole tested included:

PHMB: 0.009 wt %, 0.00048 wt %, 0.00024 wt %, 0.00012 wt %, 0.00006 wt % and 0.00003 wt %.

Miconazole: 0.00048 wt %, 0.00024 wt %, 0.00012 wt %, 0.00006 wt % and 0.00003 wt %.

PHMB/Miconazole: 0.009/0.00048 wt %, 0.009/0.00024 wt %, 0.009/0.00012 wt %, 0.009/0.00006 wt %, 0.009/0.00003 wt %, 0.00048/0.00048 wt %, 0.00048/0.00024 wt %, 0.00048/0.00012 wt %, 0.00048/0.00006 wt %, 0.00048/0.00003 wt %, 0.00024/0.00048 wt %, 0.00024/0.00024 wt %, 0.00024/0.00012 wt %, 0.00024/0.00006 wt %, 0.00024/0.00003 wt %, 0.00012/0.00048 wt %, 0.00012/0.00024 wt %, 0.00012/0.00012 wt %, 0.00012/0.00006 wt %, 0.00012/0.00003 wt %, 0.00006/0.00048 wt %, 0.00006/0.00024 wt %. 0.00006/0.0001,2 wt %, 0.00006/0.00006 wt %, 0.00006/0.00003 wt %, 0.00003/0.00048 wt %, 0.00003/0.00024 wt %, 0.00003/0.00012 wt %, 0.00003/0.00006 wt % and 0.00003/0.00003 wt %.

A drop of microbial suspension containing one of:
a) Staphylococcus pseudintermedius and
b) Candida albicans

was added to a MH agar plate and SAB plate respectively of each concentration of each concentration of miconazole, PHMB and combinations thereof and the MH plates were incubated for 24 to 48 hours at 37° C. and the SAB plates were incubated at 20 to 35° C. for 48 hours. Two isolates of Staphylococcus pseudintermedins of different origin were used.

After incubation the plates were examined to determine the lowest concentration that inhibited growth of the microbe.

The concentrations were used to calculate the Fractional Inhibitory Concentration Index (FICI) which is a measure of synergism between the components in the combination. The FICI is calculated using the following equations:

FIC_{MICONAZOLE}=MIC_{MICONAZOLE+PHMB}/MIC_{MICONAZOLE}

FIC_{PHMB}=MIC_{PHMB+MICONAZOLE}/MIC_{PHMB}

FICI=FIC_{MICONAZOLE}+FIC_{PHMB}

FICI≦0.5 Indicates synergy between components

FICI>0.5-4.0 indicates no interaction between components

FICI>4.0 indicates antagonism between components

The results are shown in Table 2.

TABLE 2

| | | | MIC | MIC | | | |
|---|---|---|---|---|---|---|---|
| | MIC | MIC | PHMB + | Micon + | FIC | FIC | |
| Microbe | PHMB % | Micon % | Micon % | PHMB % | PHMB | Micon | FICI |
| S.p | 0.00024 | 0.00012 | 0.00003 | 0.00003 | 0.125 | 0.25 | 0.375 |
| S.p | 0.00024 | 0.00012 | 0.00003 | 0.00003 | 0.125 | 0.25 | 0.375 |
| C.a | 0.0156 | 0.00095 | 0.0039 | 0.00024 | 0.25 | 0.25 | 0.5 |

S.p = Staphylococcus pseudintermedius;
C.a = Candida albicans

As can be seen from these results, the combination of PHMB and miconazole had a synergistic effect on two different isolates of Staphylococcus pseudintermedius and the yeast Candida albicans.

### Example 3

### Determination of Synergism in Treatment of Isolates from Dogs

The procedure from Example 2 was repeated with different bacterial and fungal isolates obtained from dogs with bacterial or fungal infections. Eight S. pseudintermedius isolates obtained from eight different dogs and three M. pachydermatis isolates from three different dogs were analysed. The results are shown in Tables 3 and 4.

TABLE 3
S. pseudintermedius isolates

| | MIC | MIC | MIC | MIC | FIC | FIC | | |
|---|---|---|---|---|---|---|---|---|
| Isolate | PHMB | miconazole | PHMB and miconazole | Miconazole and PHMB | PHMB | miconazole | FICI | Interaction |
| 1 | 0.00048% | 0.00012% | 0.00003% | 0.00003% | 0.06 | 0.25 | 0.31 | Synergy |
| 2 | 0.00024% | 0.00012% | 0.00003% | 0.00003% | 0.125 | 0.25 | 0.38 | Synergy |
| 3 | 0.00048% | 0.00012% | 0.00003% | 0.00003% | 0.06 | 0.25 | 0.31 | Synergy |
| 4 | 0.00048% | 0.00012% | 0.00003% | 0.00003% | 0.06 | 0.25 | 0.31 | Synergy |
| 5 | 0.00024% | 0.00012% | 0.00003% | 0.00003% | 0.125 | 0.25 | 0.38 | Synergy |
| 6 | 0.00048% | 0.00012% | 0.00003% | 0.00003% | 0.06 | 0.25 | 0.31 | Synergy |
| 7 | 0.00048% | 0.00012% | 0.00003% | 0.00003% | 0.06 | 0.25 | 0.31 | Synergy |
| 8 | 0.00048% | 0.00012% | 0.00003% | 0.00003% | 0.06 | 0.25 | 0.31 | Synergy |

TABLE 4
M. pachydermatis Isolates

| | | | MIC | | | | | |
|---|---|---|---|---|---|---|---|---|
| | MIC | MIC | PHMB and | MIC | FIC | FIC | | |
| Isolate | PHMB | miconazole | miconazole | Miconazole and PHMB | PHMB | miconazole | FICI | Interaction |
| 1 | 0.00024% | 0.00003% | 0.0000075% | 0.0000075% | 0.03 | 0.25 | 0.28 | Synergy |
| 2 | 0.00048% | 0.00006% | 0.00006% | 0.00006% | 0.125 | 1 | 1.125 | No interaction |
| 3 | 0.00048% | 0.000015% | 0.0000075% | 0.0000075% | 0.016 | 0.5 | 0.515 | Almost Synergy |

1. A pharmaceutical or veterinary composition comprising at least one azole compound or a pharmaceutically or veterinary acceptable salt thereof and at least one polymeric biguanide compound or a pharmaceutically or veterinary acceptable salt thereof.

2. The pharmaceutical or veterinary composition according to claim 1 wherein the at least one azole compound is selected from bifonazole, butoconazole, clotrimazole, econazole, fenticonazole, isoconazole, ketoconazole, Elubiol, miconazole, omoconazole, oxiconazole, sertaconazole, sulconazole, tioconazole and mixtures thereof.

3. The pharmaceutical or veterinary composition according to claim 2 wherein the at least one azole compound is ketoconazole or miconazole or a salt thereof.

4. The pharmaceutical or veterinary composition according to claim 1 wherein the polymeric biguanide compound is a compound of the formula: 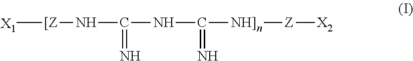
or a tautomer thereof, wherein Z is absent or an organic divalent bridging group and each Z may be the same or different throughout the polymer; n is at least 3, preferably 5 to 20 and X₁ and X₂ are independently selected from —NH₂, —NH—C(═NH)—NH—CN, optionally substituted alkyl, optionally substituted cycloalkyl, optionally substituted aryl, optionally substituted heterocyclyl and optionally substituted heteroaryl; or a pharmaceutically or veterinary acceptable salt thereof.

5. The pharmaceutical or veterinary composition according to claim 4 wherein the polymeric biguanide compound is polyhexamethylene biguanide.

6. The pharmaceutical or veterinary composition according to claim 1 wherein the polymeric biguanide compound is a compound of formula: 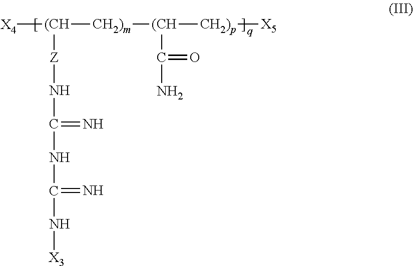
or a tautomer thereof, wherein X₃ is selected from —NH₂, —NH—C(═NH)—NH—CN, optionally substituted alkyl, optionally substituted cycloalkyl, optionally substituted aryl, optionally substituted heterocyclyl and optionally substituted aryl or a pharmaceutically or veterinary acceptable salt thereof, X₄ and X₅ are independently selected from H or X₃, Z is absent or is a divalent bridging group, m is an integer from 1 to 10, p is 0 or an integer from 1 to 10 and q is an integer from 1 to 1000.

7. The pharmaceutical or veterinary composition according to claim 1 further comprising a pharmaceutically or veterinary acceptable carrier, excipient or diluent.

8. The pharmaceutical or veterinary composition according to claim 1 wherein the composition is in the form of a gel, lotion, ointment, spray, shampoo or mousse.

9. The pharmaceutical or veterinary composition according to claim 1, further comprising an additive that enhances antimicrobial activity and/or an anti-inflammatory agent.

10. The pharmaceutical or veterinary composition according to claim 9 wherein the additive that enhances antimicrobial activity is selected from propylene glycol, glycerin, polypropylene glycol(s), polyethylene glycol(s), an antibiotic or mixtures thereof.

11. The pharmaceutical or veterinary composition according to claim 9 wherein the anti-inflammatory agent is selected from corticosteroids and non-steroidal anti-inflammatory drugs.

12. A method of treating or preventing infections of the skin or an epithelial lined cavity in a mammal, comprising topical administration of an effective amount of a composition according to claim 1.

13. The method according to claim 12 wherein the infection is a bacterial infection, a fungal infection or a mixed bacterial and fungal infection.

14. The method according to claim 12 wherein the bacterial infection is caused by a bacteria selected from one or more of a Staphylococcus spp., a Streptococcus spp. a Enterobacteriaceae spp., a Klebsiella spp., a Proteus spp., and Pseudomonads

15. The method according to claim 13 wherein the fungal infection is caused by yeast, an Aspergillus spp. or a dermatophyte.

16. The method according to claim 15 wherein the yeast is selected from Candida spp. and a Malassezia spp.

17. The method according to claim 12 wherein the infection is a skin infection.

18. The method according to claim 12 wherein the infection is an infection of the external ear.

19. A method of manufacturing a medicament for treating or preventing infections of the skin or an epithelial lined cavity in a mammal, the method comprising:
mixing the composition according to claim 1 and one or more selected from the group consisting of a pharmaceutically or veterinary acceptable carrier, excipient and diluent.
